(19) United States
(12) Patent Application Publication
(10) Pub. No.: US 2010/0036109 A1
(43) Pub. Date: Feb. 11, 2010

(54) METHOD FOR SELECTIVELY AND REVERSIBLY ADSORBING NUCLEIC ACIDS ON A CARRIER MATERIAL

(75) Inventors: Andreas Frerix, Kalkar-Kehrum (DE); Jürgen Hubbuch, Köln (DE); Markus Müller, Dormagen (DE); Michael Schönewald, Bovenau (DE)

Correspondence Address: Leon R. Yankwich;Yankwich & Associates, 201 Broadway, Cambridge, MA, 02139, US

(73) Assignee: QIAGEN GMBH, Hilden (DE)

(21) Appl. No.: 12/084,667

(22) PCT Filed: Nov. 7, 2006
(86) PCT No.: PCT/EP2006/068179

§ 371 Date: Sep. 15, 2009

(30) Foreign Application Priority Data
Nov. 7, 2005 (EP) 05024207.2

(51) Int. Cl. C07H 21/00

(52) U.S. Cl. 536/25.41

(57) ABSTRACT

The invention relates to a process for selective, reversible adsorption of nucleic acids to a support material, which process is characterized in that (a) a nucleic acid-containing mixture having a cosmotropic salt content of at least 1 mole is contacted with said support material, whereupon the nucleic acid(s) is (are) adsorbed from said nucleic acid-containing mixture to said support material, and (b) the nucleic acid(s) bound to the support material is (are) detached from the support material by means of an aqueous solution having a salt content of no more than 1 mole.

The present invention relates to a process for selective, reversible adsorption of a nucleic acid or a plurality of nucleic acids to a support material.

A problem in the purification of nucleic acids, basically unsolved to date, is that of isolating in an efficient manner nucleic acids from cosmotropic solutions with high salt concentration (cosmotropic salts are phosphates, sulfates and citrates, for example) and in particular from the lower phase of aqueous two-phase systems (polymer salt systems). Some time-consuming methods (for example ultrafiltration, gel filtration, etc.) have been applied thus far, but can be justified only in a few applications. A rapid, efficient and cost-effective precipitation of nucleic acids under such solvent conditions (for example by adding an organic solvent such as isopropanol) has not been possible thus far, since immiscibility causes a phase system to reform, leaving parts of the nucleic acids subsequently as an incomplete precipitate in the phase interface. In addition, precipitations are usually work-up techniques which are difficult to scale up. For this reason, nucleic acid-containing phases are rebuffered or formulated in the prior art using ultra-filtration, gel filtration or other suitable methods based on column-chromatographic processes.

There are also known processes for purifying double-stranded DNA (mainly plasmid DNA) by the “aqueous two-phase system” (ATPS) which is described, for example, in WO2004/106516 A1 and Trinidade, I. P., Diogo M. M., Prazeres D. M. F. and Marcos J. C., Purification of plasmid DNA vectors by aqueous two-phase extraction and hydrophobic interaction chromatography, Journal of Chromatography A, 1082 (2005), 176-184. The latter ATPS process involves further purification of the product-containing lower phase by means of conventional hydrophobic interaction chromatography, achieving purification by way of selective adsorption of an undesired nucleic acid. In this case, the product nucleic acid is not bound and therefore also not concentrated or isolated from the high salt phase.

The abovementioned column-chromatographic methods of the prior art (in particular hydrophobic processes) are disadvantageous mainly in that they entail high costs, have lower flow rates and therefore low productivity, exhibit high pressure drops, have only low capacities and require elution conditions comprising high salt concentrations which in turn result in waste disposal problems. Simultaneous desalting and separation of RNA/DNA is not possible, since RNA binds more tightly than DNA to the adsorbent in all known cases. Ultra- or diafiltration is very time-consuming, has low productivity and only low selectivity for different nucleic acid populations such as plasmid DNA, sheared genomic DNA and long-chain RNA molecules. Finally, gel filtration is disadvantageous in that high product dilution and low productivity must be accepted due to the low amounts to be applied.

It is therefore the object of the present invention to provide an improved process for selectively removing nucleic acids from aqueous systems containing cosmotropic salts, such as, for example, two-phase systems. This object is achieved according to the invention by the process as claimed in independent claim 1. Further advantageous embodiments, aspects and details of the invention arise from the dependent claims, the description, the example and the drawing.

The present invention therefore describes a process for selective, reversible adsorption of nucleic acids to a support material, which process is characterized in that (a) a nucleic acid-containing mixture having a cosmotropic salt content of at least 1 mole is contacted with said support material, whereupon the nucleic acid(s) is (are) adsorbed from said nucleic acid-containing mixture to said support material, and (b) the nucleic acid(s) bound to the support material is (are) detached by means of an aqueous solution having a salt content of no more than 1 mol, in particular less than 1 mol. Preference is given to detaching the nucleic acid from the support material (step (b) above) by using a solution having a salt concentration of 500 mM or less, for example 100 mM or less, 50 mM or less, 20 mM or less, or 10 mM or less. Further preference is given to the solution for detaching a nucleic acid from the support material having a salt concentration of at least 1 mM. The cosmotropic salt content of the solution in step (a) in which nucleic acid(s) is (are) bound to the support material is preferably at least 1.5 mol, more preferably at least 2.0 mol, and in particular at least 2.5 mol. Less preference is given to cosmotropic salt contents of more than 5 mol and in particular of more than 10 mol.

Cosmotropic (or lyotropic) salts are classified in the “Hofmeister Series” according to their property of increasing the structure of water, contrary to chaotropic salts which reduce (interfere with) water structure. Examples of known cosmotropic salts are phosphates, sulfates and citrates.

The type of adsorption described herein utilizes hydrophobic or mixed hydrophobic-electrostatic interactions (“mixed mode”). The strength of this binding—i.e. the synergistic interplay between surface properties of the adsorber, the adsorbent and the liquid phase conditions—determines the reversibility of binding desired in the process. Said binding therefore differs from the binding of nucleic acids under chaotropic conditions to silica surfaces, described in the “Boom patent” (U.S. Pat. No. 5,234,809, European patent No. 0 389 063).

Furthermore, the type of binding differs markedly from common, exclusively hydrophobic types of binding described in the literature, since the order of adsorption of DNA and RNA is reversed in the present case, i.e. that DNA (as target nucleic acid) binds more tightly than RNA. This enables the adsorbents to be utilized by DNA at greater capacity, RNA to be eluted initially and DNA to be eluted finally under desired buffer conditions with low salt concentrations. It is therefore possible to purify and desalt the sample at the same time. Binding selectivities may furthermore be modulated by combining or adding organic solvents such as ethanol or isopropanol.

“Target nucleic acid” and “target nucleic acids” mean in the context of the present application a nucleic acid and a plurality of nucleic acids, respectively, which are to be removed from a mixture of various substances, for example the components of a cell.

Particularly advantageously, the use according to the invention of this technology is suitable for capturing target nucleic acids from product-containing phases of polymer/salt phase systems, which phases have been obtained previously. Normally, the nucleic acid(s) can not be precipitated rapidly, efficiently and cost-effectively out of these solution conditions (for example by adding isopropanol), since in this case two phases consisting of the organic and the nucleic acid-containing high salt phase will form or nucleic acids will precipitate at the phase interface, see above.

The present invention therefore relates to a process for selective, reversible adsorption of nucleic acid(s) from solutions with a high concentration of preferably cosmotropic salts (“high concentration” here means in particular ≧20% w/w, for example of phosphate salts, citrate salts and sulfate salts) to a suitable support material (adsorbents, membranes, filters or monoliths), for example under hydrophobic or “mixed mode” (hydrophobic-electrostatic) conditions and subsequent desorption of said nucleic acid(s) from said support material and elution by formulation solutions with a low salt concentration of 100 mM or less, preferably 50 mM or less, and in particular 20 mM or less (for example conventional aqueous buffer systems known to the skilled worker, such as 10 mM Tris/HCl, 1 mM EDTA, pH 8.0 etc). The nucleic acids attached according to the invention may be eluted under desired aqueous or non-cosmotropic and/or low salt conditions from the adsorber matrix and used in subsequent applications. The nucleic acid target molecule is adsorbed according to the invention from high molar solutions of cosmotropic salts to suitable adsorbents, membranes, filters or monoliths, such as, for example, nitrocellulose filters and hydrophilized nylon filters, nylon pad and polyvinylidene fluoride (PVDF) and also to silica-based adsorbents. The use according to the invention of this technology is particularly suitable for capturing nucleic acids from product-containing phases of phase systems in which the target nucleic acid has been distributed in the phase containing a high concentration of cosmotropic salts, as described in WO2004/106516 A1, for example.

The process according to the invention overcomes the disadvantages of the prior art (such as time-consuming concentration and rebuffering of the nucleic acid phase, for example by ultrafiltration or gel filtration; moreover, precipitation from solutions with high concentrations of cosmotropic salts is usually not possible), facilitating the purification of nucleic acid(s) considerably. Moreover, precipitation from solutions with high concentrations of cosmotropic salts is otherwise usually not possible. Aside from rebuffering, the approach according to the invention makes possible an additional purification effect on remaining contaminants in the nucleic acid phase or DNA phase (e.g. proteins, RNA). The process according to the invention opens up applications both on the research scale and in automated high throughput analyses (e.g. in diagnostics) and in preparative applications on a production scale. Furthermore, the use of suitable support materials (e.g. adsorbents, membranes, filters or monoliths) enables high flow rates and short dwell times to be employed, since the majority of adsorption is not limited by diffusion.

The process according to the invention makes use of nucleic acid-containing solutions, preferably with a phosphate concentration of at least 20% (w/w), or nucleic acid-containing, cosmotropic, high molar sulfate solutions or citrate solutions. At lower concentrations of said cosmotropic salts, binding of the nucleic acids to the support material (adsorbent) is incomplete. Furthermore, the required final concentration of the binding-inducing, cosmotropic salt component may be modeled or optimized for the particular target nucleic acid by adding non-cosmotropic salts.

When using the present invention for isolating nucleic acid(s) from aqueous two-phase systems, the lower phase is used (as described in WO2004/106516 A1, for example). The present invention is also suitable for isolating nucleic acids from other PEG/salt phase systems described in combination with cosmotropic salts such as phosphate salts, sulfate salts and citrate salts, thereby also markedly improving for the latter the results of an isolation according to the previous prior art (for example by ultrafiltration or gel filtration).

Binding under the above-described cosmotropic conditions is possible in a particularly efficient manner and with very high capacities (up to 0.1 mg/cm² of filter area and higher) to suitable supports such as, for example, nitrocellulose filters and hydrophilized nylon filter membranes (membrane disks, filter plates, filter capsules, filter cartridges). Adsorption to PVDF can likewise take place after rinsing with isopropanol, albeit with a relatively low dynamic capacity. Although other hydrophobic membranes such as GHP or unmodified nylon cause nucleic acids to bind, bound DNA cannot be detached or eluted again completely with aqueous buffers, as it can with nitrocellulose, PVDF or hydrophilized nylon.

Suitable for binding are any nucleic acid-binding adsorbents, i.e. apart from membranes and filter materials also monoliths, resins (adsorber resins), nitrocellulose pad, granules and other formates with adsorbing properties. However, particularly suitable are materials (membrane, filters, monoliths), which have high productivity (high flow rates, low diffusion limitation).

To remove residual salt, the support material/adsorbent may be washed with an alcohol, in particular isopropanol or ethanol, preferably from 50 to 100% (v/v) alcohol or isopropanol or ethanol, particularly preferably from 60 to 90% (v/v) alcohol or isopropanol or ethanol, very particularly preferably from 65 to 85% (v/v) alcohol or isopropanol or ethanol. The nucleic acids are then in a precipitated state on the adsorbent.

The washed, desalted nucleic acid (e.g. DNA) present on the membrane may be detached and eluted from the support material in aqueous, for example buffered, solutions such as Tris/HCl-EDTA (TE), which do not result in the precipitation of DNA and moreover do not contain very high concentrations (not above 1 M) of cosmotropic salts such as sulfates, citrates and phosphates, P1 buffer (Qiagen GmbH, Hilden, Germany) or P3 buffer (Qiagen GmbH, Hilden, Germany).

The advantages of the process according to the invention are distinctly accelerated, cost-effective, scalable and automatable desalting, purification, concentration and/or rebuffering of nucleic acid-containing solutions, without the need for using complicated and limited techniques such as ultrafiltration, gel filtration, etc. Depending on the object and optimization of the conditions as a function of the particular target nucleic acid (for example plasmid DNA, genomic DNA, etc.), RNA and proteins may also be reduced markedly. The processes disclosed previously by the prior art are thus improved considerably.

The process of the invention can be carried out as follows. First, a nucleic acid-containing solution must be provided. This solution may be obtained, for example, by extracting nucleic acid-containing (e.g. DNA-containing or plasmid-containing) process liquids such as bacterial lysates, or by purification techniques such as chromatography, precipitation, filtration etc., prepurified process intermediates in a suitable phase system containing a suitable cosmotropic phase with high salt concentration, as described in WO2004/106516 A1, for example. The nucleic acid-containing (DNA-containing or plasmid-containing) phase is then immobilized by means of adsorption to a suitable support material.

Residual salt may be removed in an optional step by washing the support material with organic solvents such as, for example, alcohols such as isopropanol or ethanol. Alternatively, the nucleic acids may also be attached to the support material selectively using other solutions resulting in the precipitation of nucleic acids (e.g. PEG, spermidine, spermine, CTAB=cetyltrimethylammonium bromide).

The washed, desalted nucleic acid (e.g. DNA or plasmid) present on the membrane may then, in a further step, be detached and eluted from the support material in aqueous, for example buffered, solutions such as TE, which do not result in the precipitation of DNA and moreover do not contain very high concentrations (not above 1 M) of cosmotropic salts such as sulfates, citrates and phosphates, in P1 buffer (Qiagen GmbH) or in P3 buffer (Qiagen GmbH).

The nucleic acid(s) eluted and purified in this way is (are) available for analyses, for example in diagnostics, for further work-up steps or for therapeutic applications, for example gene therapy or genetic vaccinations.

The present invention concerns selective, reversible adsorption of nucleic acids under cosmotropic binding conditions to suitable adsorbents, for example to hydrophobic surfaces. Particular preference is given to nitrocellulose filters and nylon filters and also to PVDF as support materials or adsorbents. Particular preference is given to nucleic acids being selectively and reversibly adsorbed under cosmotropic binding conditions, after separation and extraction in phase systems, from nucleic acid-containing phases of phase systems, in particular aqueous two-phase systems, as disclosed by WO2004/106516 A1 and Trinidade, I. P. et al. (see above).

The process of the invention is particularly suitable for isolating double-stranded nucleic acids from aqueous two-phase systems or organic/aqueous two-phase systems under cosmotropic binding conditions, after separation and extraction in phase systems, from product-containing phases of phase systems to hydrophobic adsorbents such as, for example, nitrocellulose filters and nylon filters and PVDF.

A further aspect of the invention relates to isolating double-stranded nucleic acids such as, for example, plasmid DNA from the lower phase of a PEG/potassium phosphate system to hydrophobic adsorbents such as, for example, nitrocellulose filters and nylon filters and PVDF.

The invention is illustrated in more detail by an example below.

### EXAMPLE

An aqueous two-phase system (300 g) containing 6 g of biomass was treated as described in WO2004/106516 A1. The reaction mixture was admixed with 15% polyethylene glycol (PEG) 800 and 20% phosphate, and the aqueous lower phase formed was isolated. The lower phase was applied to a nitrocellulose membrane, and various batches were then analyzed by high pressure liquid chromatography (HPLC) by means of hydrophobic interaction chromatography (HIC).

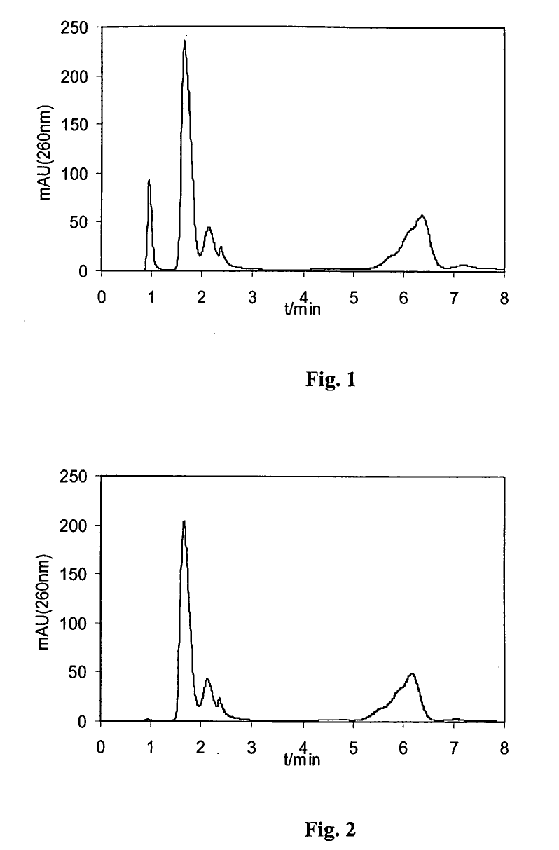

FIG. 1 to 3 depict elution profiles of three HPLC runs. The optical density at 260 nm of the particular sample is plotted on the ordinate and time in minutes is plotted on the abscissa.

FIG. 1 is an HPLC diagram (column: Source 15 PHE (GE Healthcare), elution with 1.5 M ammonium sulfate, 10 mM Tris/Cl, pH 8.0; 0.8 ml/min after 2.5 min gradient to 10 mM Tris/Cl, pH 8.0 until elution of RNA) of a sample which represents the lower phase as total fraction as obtained after aqueous two-phase separation. The sample contained 26.5 μg/ml pDNA and 57.5 μg/ml RNA. The first peak of the elution profile depicted in FIG. 1 at about 1 min is caused by the pDNA present in the sample, the next peaks, after a retention time of between 1.5 and 2.5 minutes, depict salts, proteins and oligonucleotide RNA, and the last peak which eluted from the HPLC column at between 6 and 7 minutes is that of RNA.

FIG. 2 depicts the HPLC elution profile of the lower phase, after 20 ml of said lower phase had been filtered through a nitrocellulose membrane of 47 mm in diameter and 0.2 μm pore size (30% w/w potassium phosphate buffer). The sample contained 0.5 μg/ml pDNA and 50 μg/ml RNA. As can be readily observed, the sample is free of pDNA (no corresponding peak at about 1 min) which is bound virtually quantitatively to the nitrocellulose membrane. The other peaks of FIG. 1 (salts, proteins, oligonucleotide RNA, RNA) are also present unaltered in FIG. 2. This demonstrates that pDNA has been removed from the sample selectively and quantitatively.

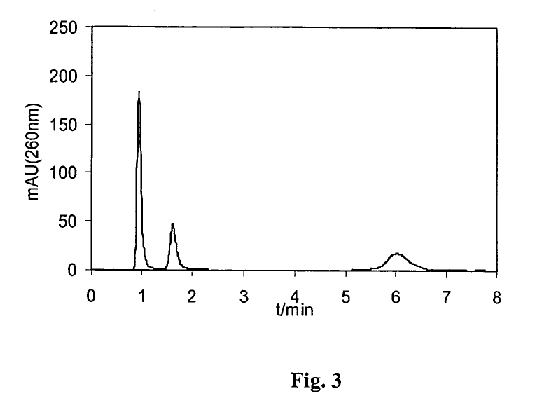

Finally, FIG. 3 depicts a sample obtained after elution of the nitrocellulose filter with 10 ml of TE buffer (10 mM Tris/HCl, 1 mM EDTA, pH 8.0). The yield of pDNA was 50 μg/ml (corresponding to approx. 95% yield; 501 μg of 530 μg of pDNA used were recovered), and the amount of RNA in the sample was 16.4 μg/ml RNA (corresponding to a reduction of approx. 86%; 1150 μg of RNA were used, 164 μg were found again in the eluate). This demonstrates that pDNA can be recovered almost completely in a highly purified form after appropriate treatment (binding to a support material, in the present case to a nitrocellulose filter).

What is claimed is:

1. A process for selective, reversible adsorption of nucleic acids to a support material, comprising:
(a) contacting a nucleic acid-containing mixture having a cosmotropic salt content of at least 1 mole with said support material, whereupon the nucleic acids are adsorbed from said nucleic acid-containing mixture to said support material, and
(b) detaching the nucleic acids bound to the support material by means of an aqueous solution having a salt content of no more than 1 mole.

2. The process as claimed in claim 1, characterized in that the support material is selected from the group consisting of a membrane, a filter, a nylon pad, and polyvinylidene fluoride (PVDF).

3. The process as claimed in claim 2, characterized in that the filter is a nitrocellulose filter or a hydrophilized nylon filter.

4. The process as claimed in claim 1, characterized in that the support material consists of silica surfaces.

5. The process as claimed in claim 1, characterized in that the cosmotropic salt content of said mixture in step (a) is greater than 1 molar.

6. The process as claimed in claim 5, characterized in that the cosmotropic salt is selected from the group consisting of sulfate, citrate, and phosphate salts.

7. The process as claimed in claim 1, characterized in that the aqueous solution having a salt content of no more than 1 mole, in step (b), is a TE buffer (Tris/HCl-EDTA buffer) or water.

8. The process as claimed in claim 7, characterized in that the TE buffer comprises 10 mM Tris/HCl and 1 mM EDTA and has a pH of between 7 and 8.

9. The process as claimed in claim 1, further comprising a purification step using an organic solvent or a precipitant for nucleic acids, said solvent or precipitant selected from the group consisting of spermidine, spermine, CTAB, and PEG, and wherein said purification step is carried out after step (a) and before step (b).

10. The process as claimed in claim 9, characterized in that the organic solvent is an alcohol.

11. The process as claimed in claim 1, characterized in that it is carried out after an aqueous two-phase separation of nucleic acids.

12. The process as claimed in claim 10, characterized in that said alcohol is selected from the group consisting of ethanol and isopropanol.
